(19) United States
(12) Patent Application Publication
(10) Pub. No.: US 2006/0099690 A1
(43) Pub. Date: May 11, 2006

(54) Extraction, purification and conversion of flavonoids from plant biomass

(75) Inventors: Peter R Chang, Saskatoon (CA); Alister Muir, Saskatoon (CA)

Correspondence Address: FROST BROWN TODD, LLC, 2200 PNC CENTER, 201 E. FIFTH STREET, CINCINNATI, OH, 45202, US

(73) Assignee: Her Majesty the Queen in Right of Canada, as Repre, Ottawa (CA)

(21) Appl. No.: 10/528,877

(22) PCT Filed: Sep. 23, 2003
(86) PCT No.: PCT/CA03/01453

§ 371 Date: Mar. 23, 2005

Related U.S. Application Data
(60) Provisional application No. 60/413,002, filed on Sep. 23, 2002.

(51) Int. Cl. C12P 19/60; A61K 31/7048

(52) U.S. Cl. 435/75; 514/27

(57) ABSTRACT

A process for preparing a rutin-enriched composition from plant biomass comprises extraction with an aqueous solution, and precipitation. An enzyme preparation, such as naringinase, is used for the transformation of rutin to higher value compositions containing increased proportions of isoquercitrin and quercetin.

### REFERENCES

Abou-Karam, M., and Shier, W. T. 1992. Isolation and characterization of an antiviral flavonoid from Waldsteinia fragarioides. J. Nat. Prod. 55: 1525-1527.

Agullo, G., Gamet, L., Besson, C., Demigne, C., and Remesy, C. 1994. Quercetin exerts a preferential cytotoxic effect on active dividing colon carcinoma HT29 and CaCa-2 cells. Cancer Letters. 87: 55-63.

Agullo, G., Gamet-Payrastve, L., Manenti, S., Viala, C., Remesy, C., Chap, H., and Payrastve, B. 1997. Relationship between flavonoid structure and inhibition of phosphatidylinositol 3-kinase: a comparison with tyrosine kinase and protein kinase C inhibition. Biochem. Pharmacology. 53: 1649-1657.

Anonymous. 1990a. Hayashibara Biochemical: To sample distribute high-concentration water-soluble rutin. New Technology Japan. February. Pp 34.

Anonymous. 1990b. Water soluble rutin functioning as Vitamin P. Japan Report Medical Technology. February.

Arata, A. 1992. External Agent for Skin. Japanese Patent JP6128142 (issued May 10, 1994).

Ashida, H., Fukuda, I., Yamashita, T., and Kanazawa, K 2000. Flavones and flavonoids at dietary levels inhibit a transformation of aryl hydrocarbon receptor induced by dioxin. FEBS Letters. 476: 213-217.

Backhaus, E. 1995a. Use of bioflavonoids such as rutin or quercetin to inhibit protease enzymes, which promote aging. German Patent DE4339486 (issued Jul. 05, 1995).

Backhaus, E. 1995b. Use of bioflavonoid, especially rutin for retrovirus inactivation. Germany Patent DE4340438 (issued Jun. 01, 1995).

Balandina, I. A., Glyzin, V. I., Grinkevich, N. I., Gorodestakii, L. S., Kristall, Z. B., Shemerenkin, B. V. 1982. Rutin Production Russian Patent SU904709 (issued Feb. 15, 1982).

Basarkar, P. W. 1981. Cholesterol lowering action of vitamin P-like compounds in rats. Indian J. Exp. Biol. 19: 787-9.

Borrelli, F., and Izzo, A. A. 2000. The plant kingdom as a source of anti-ulcer remedies. Phytother. Res. 14: 581-591.

Bors, W., Heller, W., and Saran, M. 1990. Flavonoids as antioxidants determination of radical-scavenging efficiencies. Methods Enzymol. 186:343-355.

Caltagiron, S., Rossi C., Poggi, A., Ranelletti, F. O., Natali, P. G., Bruneti, M., Aiello, F. B., and Piantelli, M. 2000. Flavonoids apigenin and quercetin inhibit melanoma growth and metastatic potential. Int J. Cancer. 87: 595-600.

Crespy, V., Morand, C., Besson, C., Demigne, C., and Remesy, C. 1999. Part of quercetin absorbed in the small intestine is conjugated and further secreted in the intestinal humen. Am. J. Physiol. 277: G120-126.

Deschner, E. E. 1992. Dietary quercetin and rutin: Inhibitors of experimental colonic neoplasia. Pp 265-268. In Phenolic Compounds in Food and their Effects of Health II: Antioxidants and Cancer Prevention; Hnang, M -T., Ho, C -T., Lee, C. Y., Eds.; American Chemical Society Washington, D.C.

Ferry, D. R., Smith, A., Malkhandi, J., Fyfe, D. W., Detakats, P. O., Anderson, D., Baker, J., and Kerr, D. J. 1996. Phase I clinical trial of the flavonoid quercetin: Pharmacokinetics and evidence for in vivo tyrosine kinase inhibition Clin. Cancer Res. 4: 659-668.

Gee, J. M., Dupont, M. S., Day, A. J., Plumb, G. W., Williamson, G., and Johnson, I. T. 2000. Intestinal transport of quercetin glycosides in rats involves both deglycosylation and interaction with hexose transport pathway. J. Nut 130: 2765-2771.

Griffith, J. Q., Couch, J. F., Lindauer, M. A. 1944. Effects of rutin on increased capillary fragility in man. Proc. Soc. Exptl. Biol. Med. 55: 228-229.

Griffiths, L. A. and Barrow, A. 1972. Metabolism of flavonoid compounds in germ-free rats. Biochem. J. 130: 1161-2.

Harbone, J. B. 1994. The flavonoids. Advances in Research Since 1986. Chapman and Hall, London. P676.

Heywang, U. and Basedow, A. 1992. Extraction of rutin from plants with dioxane. German Patent DE4107079 (issued 1992-09-10).

Humphreys, F. R. 1964. The occurrence and industrial production of rutin in southeastern Australia. Economic Botany. 18: 195-253.

Huo, X. 1999. Production process of extracting rutin from Polygonum tataricum. Chinese Patent CN1217329 (issued May 06, 1999).

Iwata, K, Miwa, S., Inayama, I., Sasaki, H., Soeda, K, Sugahara, T. 1990. Effects of kangra buckwheat on spontaneously hypertensive rats. J. Kagawa Nutr. Coll. 21: 5S-61.

Ishige, K, schubert, D., and Sagara, Y. 2001. Flavonoids protect neuronal cell from oxidative stress by three distinct mechanisms. Free Radical Biology & Medicine. 30(4): 433-446.

Kato, N., Tosu, N., Doudou, T., Imamura, T. 1983. Effects of dietary quercetin on serum lipids. Agric. Biol. Chem. 47: 2119-20.

Kitabayashi, H., Ujihara, A., Hirose, T., Minami M. 1995. Varietal differences and heritability for rutin content in common buckwheat, Fagopyrum esculentum Moench. Jpn. J. Breed. 45: 75-79.

Liu, C. 1991. Extraction of therapeutic rutin from Sophora japonica buds. Chinese Patent CN1013579 (issued Aug. 21, 1991).

Lutterodt, G. D., and Abu Raihan, S. M. 1993. Calcium modulation and antinociceptive efficacy of quercetin compounds. Asia Pacific J. of Pharmacology 8:127-131.

Manach, C., Morand, C., Crespy, V., Demigne, C., Texier, O., Regerat, F., Remesy, C. 1998. Quercetin is recovered in human plasma as conjugated derivatives which retain antioxidant properties. FEBS Letter. 426:331-336.

Manach, C., Morand, C., Demigne, C., Texier, O., Regerat, F., and Remesy, C. 1997. Bioavailability of rutin and quercetin in rats. FEBS Letters. 409: 12-16.

Manach, C., Regerat, F., Texier, O., Agullo, G., Demigne, C., and Remesy, C. 1996. Bioavailability, metabolism and physiological impact of 4-oxo-flavonoids. Nutr. Research 16(3): 517-544.

Matsubara, Y., Kumamoto, R, Iizuka Y., Murakami, T., Okamoto, K, Miyake, H., Yokoi K. 1985. Structure and hypotensive effect of flavonoid glycosides in citrus unshiu peelings. Agric. Biol. Chem. 49: 909-914.

Matsumoto, T. and Hamamoto, T. 1990. Recovery of flavonoid compounds from plant extracts. Japanese Patent JP02073079 (issued Mar. 13, 1990).

Middleton, E. Jr. and Kandaswami, C. 1993. The impact of plant flavonoids on mammalian biology: implications for immunity, inflammation and Cancer. In: The flavonoids: Advances in Research since 1986. Pp 619-652. Editor: J. H. Harborne, Alan R. Liss. New York

Midori, I. 1994. Health beverage to promote alcohol metabolism—contains quercetin glycoside, divalent metal ions and liquorice extract Japanese Patent JP06248267 (issued Sep. 06, 1994).

Minami M., Kitabayashi, R, and Ujihara, A. 1998. Quantative analysis of rutin in buckwheat (Fagopyrum sp.) by high performance liquid chromatography. J. of the Faculty of Agriculture Shinshu University. 34(2): 91-95.

Morand, C., Crespy, V., Manach, C., Besson, C., Demigne, C., and Remesy, C. 1998. Plasma metabolites of quercetin and their antioxidant properties. Amer. J. Physiol. 3275(1 pt 2): R212-219.

Morand, C., Manach, C., Crespy, V., and Remesy, C. 2000. Quercetin 3-o-beta-glucoside is better absorbed than other quercetin forms and is not present in rat plasma. Free Radical Research. 33(5): 667-676.

Nakayama, T. 1994. Quercetin, kaempferol, catechin or taxifolin as antioxidant—for use in food or as pharmaceutical, e.g for treating ischaemia, rheumatism or diabetes. Japanese Patent JP06248267 (issued Sep. 06, 1994).

Narikawa, T., Karaki, Y., Shinoyama, H, and Fuji T. 1998. Rutin Degradation by culture filtrates from Penicillia. Nippon Nogeikagaku Kaishi 72(4): 473-479

Nishimura, M., Horikawa, H., Moriwaki, M. 1992. Composition and process for dissolving a sparingly water soluble flavonoid. U.S. Pat. No. 5,122,381 (issued Jun. 16, 1992).

Noroozi, M., Angerson, W. J., and Lean, M. E. J. 1998. Effects of flavonoids and vitamin C on oxidative DNA damage to human lymphocytes. Am. J. Clinical Nutr. 67(6): 1210-1218.

Okuyama, T., Sato, H., Nomura, K. 1996. Extraction of blood platelet aggregation-inhibition quercetin or its glycoside from Alpinia urarensis hay. Japanese Patent 0734037 (issued Dec. 12, 1992).

Oomah, B. D. and Mazza, G. 1996. Flavonoids and antioxidant activity in buckwheat. J. Agric. Food Chem. 44: 1745-1750.

Pisha, E., Pezzuto, J. M. 1994. Fruits and vegetables containing compounds that demonstrate pharmacological activity in humans. In Economic and Medical Plant Research, Vol. 6.; Wagner, H., Hikino, H., Farnsworth, N. R., Eds.; Pp 189-233. Academic Press, London, UK.

Prochazka, V. 1985. Can wild buckwheat be a commercial source of rutin? Nase Liecive Rastliny 22(5): 131-133.

Skibola, C., and Smith, M. T. 2000. Potential Health impacts of excessive flavonoid intake. Free Radical Biology & Medicine. 29(3/4): 375-383.

Sloley, B. D., Urichuk, L. J., Ling, L., Gu, L. -D., Coutts, R. T., Pang, P. K. and Shan, J. J. 2000. Chemical and pharmacological evaluation of Hypericum perforatum extracts. Acta Pharmacol. Sin. 21(12): 1145-1152.

Suzuki, Y., Suzuki, K., Yoneyama, M., Hijiya, H., Miyaka, T. 1992a Preparation and uses of alpha-glycosyl rutin. U.S. Pat. No. 5,145,781 (issued Sep. 08, 1992).

Suzuki, Y., Suzuki, K., Yoneyama, M., Hijiya, H., Miyaka, T. 1992b. 4alpha-D-glucopyranosyl rutin and its preparation and uses. U.S. Pat. No. 5,171,573 (issued Dec. 15, 1992).

Suzuki, Y., Suzuki, K., Yoneyama, M., Hijiya, H. 1995. Preparation and uses of alpha glycosyl rutin. European Patent 0387 042 B1 (issued Feb. 15, 1995).

Suzuki, Y., Suzuki, K., Yoneyama, M., Miyaka, T. 1996. 4alpha-D-glucopyranosyl rutin and its preparation and uses. European Patent 0420376 B1 (issued Mar. 13, 1996).

Uyeta, M., Taue, S., and Mazaki, M. 1981. Mutagenicity of hydrolysates of tea infusions. Mutation Research 88: 233-240.

Valerio, L. G., Kepa, J. K., Pickwell, G. V., and Quattrochi, L. C. 2001. Induction of human NAD(P)H: quinone oxidoreductase (NQO1) gene expression by the flavonol quercetin. Toxicology Letters 119:49-57.

Wang, H -K. 2000. The therapeutic potential of flavonoids. Exp. Opin. Invest. Drugs 9(9): 2103-2119.

Washino, K. 1992. The manufacture of water-soluble flavonol glycosides with galactosidase and glucanotransferase. Japanese Patents 04066098 and 04066096 (issued Mar. 02, 1992).

Watanabe, J., Kawabata, J., Kurihara, H., and Niki, R. 1997. Isolation and identification of alpha-glucosidase inhibitors from Tochu-cha (Eucommia ulmoides). Biosci. Biotech. Biochem. 61(1): 177-178.

Yesilada, E., Tsuchiya, K., Takaishi, Y. and Kawazoo, K. 2000. Isolation and characterization of free radical scavenging flavonoid glycosides from the flower of Sparticm junceum by activity-guided factionation J. of Ethnopharmacology. 73: 471-478.

Yildzogle-Ari, N., Altan, V. M., Altinkurt O., Ozturk, Y. 1991. Pharmacological effects of rutin. Phytotherapy Res. 5: 9-23.

Yoneyama, M., Iritani, S., Miyake, T. 1996. Alpha-glycosyl quercetin and its preparation and uses. U.S. Pat. No. 5,565,435 (issued Oct. 15, 1996).

Zhai, G. 1997. Preparation of rutin by continuous extraction. Chinese Patent CN1160048 (issued Sep. 24, 1999).

Zirlin, A. D. 1974. Prevention of crystallizaton of sparingly soluble flavonoids in food systems. U.S. Pat. No. 3,832,475 (issued 1974-8-27).

This invention relates to flavonoids and in particular to rutin enriched compositions prepared from plant biomass, which can be enzymatically converted to the more valuable flavonoids isoquercitrin and quercetin

### BACKGROUND OF THE INVENTION

Plant flavonoids usually occur in plants as glycosides, although in some circumstances they may occur as free aglycones. Most glycosides are O-glycosides, with the most common monoglycoside being at the 7-position. Diglycosides usually have sugars at the −7 and −3 positions and occasionally the −7 and −4′ positions. Other combinations and mono-glycosides exist but are less abundant. C-glycosides also occur in a more restricted distribution with C-6 and C-8 glycosides being the most common (Harbone, 1994).

Plant flavonoids have antioxidative properties (Bors et al., 1990), cytostatic effects in tumorigenesis, and the ability to inhibit a broad spectrum of enzymes, such as angiotensin converting enzyme (ACE), protein kinase C, tyrosine protein kinase, and topoisomerase II. They are regarded as potential cancer preventatives and cardioprotective agents (Manach et al., 1996; Skibola and Smith, 2000). Their potential use as anti-inflammatory or antiviral agents has also been examined (Middleton and Kandaswami, 1993). Backhaus (1995a) claimed that bioflavonoids, especially rutin, citrin, quercetin, hesperidin or derivatives were responsible for the inactivation of protein-cleaving enzymes (such as hyaluronidase and/or collagenase), which promote skin-aging processes. These compounds may be used for general skin care or cosmetic surgery. It is reported that rutin, quercetin, isoquercitrin, catechin and other compounds also prevent and ameliorate the aging phenomena of the skin (Arata, 1992). Midori (1994) claimed that, together, quercetin glycoside, divalent metal ion, and extract of liquorice prevent intoxication by promoting alcohol metabolism in the human liver.

Rutin is a flavonoid glycoside comprised of quercetin and the sugar, rutinose. Many beneficial health effects of rutin have been demonstrated. Such effects have been attributed to ant-inflammatory, anti-mutagenic, anti-tumor, anticarcinogenic, smooth muscle relaxation, and estrogen receptor binding activities of rutin (Pisha and Pezzuto, 1994). Rutin is also being used in the treatment of capillary fragility, cerebral thrombosis, retinitis and rheumatic-fever-associated haemorrhagic conditions (Griffith et al., 1944; Matsubara et al., 1985; Iwata et al., 1990; Yildzogle-Ari et al., 1991). Under conditions of low dietary fat intake, rutin and quercetin have been reported to considerably suppress colon tumor incidence (Agullo et al., 1994; Deschner, 1992). Backhaus (1995b) claimed that rutin and its derivatives, in an oral dosage form, and injection or infusion solution, or a suppository, would inactivate retroviruses (e.g. is HIV). Rutin can be used as a natural coloring agent an oxidation inhibitor, vitamin, sunburn preventative in cosmetics (rutin will absorb ultra violet rays), and as an ingredient in functional food applications (Anonymous, 1990a,b). Rutin can be found in many plants including buckwheat (leaves, flowers, stems, straws, hulls, and groats), Japanese pagoda tree (Sophora japonica), tomatoes, pansies (Viola sp., Violaceae), tobacco, forsythia, hydrangea, fava d'anta (Dimorphandra gardnerina and Dimorphandra mollis) and eucalyptus (Humphreys, 1964). Buckwheat is considered to be capable of providing a major dietary source of rutin. Kitabayashi et al. (1995) reported that the rutin content of buckwheat seed ranges from 0.126 to 0.359 mg/g dry weight Oomah and Mazza (1996) reported 0.47 and 0.77 mg/g dry weight of rutin in whole seed and hull, respectively. They also reported that flavonoids were highly concentrated in the hulls; the mean flavonoid content of buckwheat seeds and hulls were 3.87 and 13.14 mg/g, respectively. Prochazka (1985) reported that 6% rutin (wt/wt) was found in carefully dried, Czechish buckwheat leaves at the flowering stage. Dry herbage yields were 600 to 1000 Kg/ha, which at 4% (wt/wt) rutin concentration, amounted to 24-40 kg rutin/ha.

Although most of the details of the industrial production of rutin are proprietary and not described in the open literature, we are aware that Merck GmbH extracts rutin from fava d'anta for commercial purposes. Heywang and Basedow (1992) of Merck GmbH Germany, extracted rutin from shoots of fava d'anta (Dimorphandra) with 1,4-dioxane under reflux. Rutin was recovered by crystallization at room temperature. Dioxane is, however, considered carcinogenic.

Huo (Chinese Patent 1217329, 1999) described an extraction of rutin from tartary buckwheat seeds by washing with water, coarse grinding, coarse screening, soaking in water, drying in the air, fine grinding, soaking in edible alcohol, extracting below 60° C., and filtering. Balandina et al. (1982) extracted rutin from buckwheat seeds with hot water to remove the desired product and crystallized it.

Zhai (Chinese Patent CN 1160048, 1997) described the extraction of rutin from Flos sophorae by soaking with saturated limewater containing 1-10% borax, and precipitating at pH 1-6 by adding HCl.

Matsumoto and Hamamoto (1990) recovered rutin from Sophora augustifolia buds with methanolic extraction, adsorption onto activated carbon followed by desorption, by elution with 1% ammonia in 40% ethanol, and recrystallization from 20% ethanol.

Liu (1991) described a method of extracting rutin from Japanese Pagoda tree (Sophora japonica) buds by pulverizing, streaming in limewater, neutralizing the supernatant, cooling, filtering, washing, and drying the precipitates. The yield was 14.2% (wt/wt) and the product contained 95.1% (wt/wt) rutin.

Sloley et al. (2000) reported that, while hypericin is regarded as a marker chemical for extracts of leaves and flowers of Hypericum perforatum (St. John's wort), other compounds such as hyperforin, hyperoside, rutin and quercetin are presented in much higher concentrations. They also found that chemical composition profiles varied greatly among different extracts. However, free-radical-scavenging capacity correlated positively to quercetin content. The averaged rutin and quercetin contents in sixteen St. John's wort extracts were 2.0 and 0.3% (wt/wt), respectively.

One gram of rutin can be dissolved in about 8 L of water at room temperature or 200 ml of boiling water. Zirlin (U.S. Pat. No. 3,822,475, 1974) disclosed a method for prevention of crystallizaton of sparingly soluble flavonoids in acidic soft drinks. Flavonoids are mixed with sucrose and heated to a caramel melt stage (140-185° C.), dissolved in an aqueous system, and the water is evaporated off to obtain the dry mixture.

Quercetin glycosides were modified to water-soluble flavonol glycosides with alpha-glucosidase (E.C. 3.2.1.20), cyclomaltodextrin glucanotransferase (E.C. 2.4.1.19), alpha-amylase (E.C. 3.2.1.1), glucoamylase E.C. 3.2.1.3), beta-amylase (E.C. 3.2.1.2), and galactose-transferring enzymes (β-galactosidases) as described by San-Ei Chemical Industries Ltd. and Hayashihara Biochemical Laboratory Inc. (Nishimura et al., 1992; Suzuki et al., 1992a,b; Suzuki et al., 1995; Suzuki et al., 1996; Washino 1992; Yoneyama et al., 1996). Hayashihara Biochemical Laboratory Inc. claimed that they succeeded in producing water-soluble rutin which water solubility was increased by more than five thousand times (Anonymous, 1990a).

Before 1990, quercetin was considered to be mutagenic and carcinogenic (Manach et al., 1996). Metabolic animal studies have shown that quercetin may be rapidly converted to the non-mutagenic 3′-O-methylquercetin (Morand et al., 1998; Skibola and Smith 2000). More importantly, quercetin is reported to have antibacterial, antiviral, antioxidant, antiproliferative, ant-inflammatory, and anticarcinogenic effects (Crespy et al., 1999; Skibola and Smith, 2000).

Quercetin has also shown powerful inhibitory activity on various tumor cells (Middleton and Kandaswami, 1993; Caltagirone et al., 2000), colon cancer cells (Agullo et al., 1994; Deschner, 1992) and ulcers (Borelli and Izzo, 2000). Quercetin has been identified as a potent topoisomerase II inhibitor at low concentrations, similar in activity to the epipodophylotoxins widely used in cancer therapy (Skibula and Smith, 2000).

Ishige et al. (2001) showed that many flavonoids and related polyphenolic compounds protected the mouse hippocampal cell line HT-22 and rat primary neurons from oxidative stress caused by glutamate. This finding is significant because nerve cell death from oxidative stress has been implicated in a variety of pathologies, including stroke, arteriosclerosis, trauma, and Alzheimer's and Parkinson's diseases. Their data show that some flavonoids (quercetin, kaempferol, and fisetin) are quite protective, while others (rutin, chrysin, and apigenim) are inactive. Quercetin alters glutathione (GSH) metabolism and inhibits reactive oxygen species (ROS) in a cell culture model of oxidative stress. Its mechanism of action is similar to that of propyl gallate and methyl caffeate, but different from that of vitamin E. Noroozi et al. (1998) reported that quercetin is more potent than rutin and vitamin C in countering against oxidative DNA damage.

Ashida et al. (2000) reported that dietary flavonols (quercetin and rutin) and flavones suppress antagonistically the transformation of aryl hydrocarbon receptor (AhR) induced by dioxin. Quercetin is more potent that rutin in counteracting the toxicity of this environmental contaminant. In the area of anticarcinogenicity, phase I enzymes oxidize, reduce or hydrolyze carcinogens, and phase II enzymes conjugate or otherwise affect carcinogens. Valerio et al. (2001) demonstrated that quercetin is a phase II enzyme inducer that stimulates phase II detoxifying activities. Phase II enzymes can also scavenge strong oxidants, and scientific interest has been directed toward their activity as a means of decreasing the risk of cancer. Use of phase II enzyme inducers, many of which are found in common foods, is one way to increase phase II enzyme activities in body tissues.

Agullo et al. (1997) reported that quercetin was an effective inhibitor of phosphatidyl inositol 3-kinase (PI 3-kinase; an enzyme involved in cell multiplication and transformation). Luteolin, apigenin and myricetin also exhibit such activity. Inhibition of PI 3-kinase may be linked to the antitumor properties of these flavonoids. Also, quercetin was reported to inhibit lymphocyte tyrosine kinase activity, and exhibit antitumor properties in a phase I clinical trial (Ferry et al. 1996).

Watanabe et al. (1997) reported that quercetin is responsible for the alpha-glucosidase inhibitor activity of tochu (Eucommia ulmoides) leaves. Since alpha-glucosidase is an enzyme that catalyzes a final step in the digestive process of carbohydrates, the implication of the above finding is that quercetin may suppress postprandial hyperglycemia and could be used for the treatment of diabetes with potential application of late diabetic complication, obesity and related disorders. Quercetin also blocks an enzyme that leads to accumulation of sorbitol which has been linked to nerve, eye and kidney damage in those with diabetes. However, no human research has evaluated the possible beneficial effect of quercetin for diabetics (Wang, 2000).

Kato et al. (1983) showed that in mice or rats receiving 0.5% quercetin in their diets there was a significant lowering of serum triglycerides. Supplementation of quercetin was also shown effective in blunting the rise of serum and liver cholesterol in rats fed a high cholesterol diet (Basarkar, 1981)

Quercetin and its glycoside extracted and purified from the leaf of Alpinia urarensis Hay showed blood platelet aggregation-inhibition activity. Its activity was greater than that of aspirin or ginseng saponins as control blood platelet aggregation inhibitors (Okuyama et al., 1996).

In Japanese patent publication No. 06248267, Nakayama (1994) claimed that quercetin, kaempferol, catechin or taxifolin can be used in food or as pharmaceuticals for prevention and treatment of diseases caused by malfunction or scavenging action, ischaemic disease, rheumatism, diabetes etc.

Lutterodt and Abu Raihan (1993) reported that quercetin has narcotic-like antinociceptive activity that interferes with pain transmission. A dose of 50 mg of quercetin/Kg body weight would have the same effect as that of 2.5 mg of morphine sulfate/Kg.

Naturally occurring isoquercitrin (quercetin-3-O-beta glycoside) can be extracted from flowers of levant cotton (Gossypium herbaceum), Waldsteinia fragarioides (Michx) Tratt (Rosaceae), Spartium junceum L. (Fabaceae) (Yesilada et al., 2000), and horse chestnut (Aesculus hippocastanum). It is also found in celery seed, fennel seeds, horsetail, red clover and St. John's wort. Isoquercitrin has shown to possess several biological activities, including inhibition of angiotensin converting enzyme (ACE), inhibition of prostaglandin synthesis, and antiviral activity (Abou-Karam and Shier, 1992).

The role of bacterial enzymes in the digestive absorption of flavonoids is important because mammalian tissues are unable to synthesize such hydrolases. Griffiths and Barrow (1972) have shown that flavonoid glycosides ingested by germ-free rats were recovered unhydrolyzed in the feces. Hydrolysis of the sugar-aglycone bond takes place in the distal ileum and the caecum.

During absorption across the intestinal membrane, flavonoids are absorbed in the aglycone and/or glucoside forms and are partly transformed into their glucuronides, sulfates or methoxylates (Manach et al., 1998). Free quercetin could not detected in blood plasma. The small fraction of flavonoids that is absorbed is metabolized by liver enzymes resulting in polar conjugates being excreted in the urine or returned to the duodenum via the gallbladder. The largest fraction of ingested flavonoids, that is not absorbed, is degraded by the intestinal microflora. The bacterial enzymes catalyze several reactions, including hydrolysis, cleavage of the heterocyclic oxygen-containing ring, dehydroxylation, and decarboxylation. Several phenolic acids are produced, depending on the structure of the flavonoid involved. Phenolic acids can then be absorbed and subjected to conjugation and O-methylation in the liver and may then enter into the circulation (Manach et al., 1996).

Crespy et al. (1999) demonstrated that quercetin and isoquercitrin are much more bioavailable than rutin. Rutin is absorbed more slowly than quercetin, isoquercitrin and isorhamnetin because it must be hydrolyzed by the caecal microflora, whereas quercetin, isoquercitrin and isorhamnetin are absorbed from the small intestine (Manach et at., 1997). Morand et al (2000) also showed that isoquercitrin is better absorbed than other quercetin forms (quercetin, rutin and quercitrin). Four hours after a meal, the metabolites identified in hydrolyzed plasma were 3′- and 4′-methylquercetin regardless of what form of quercetin was consumed. However, the total concentration of metabolites in the plasma was markedly different 33.2, 11.2, and 2.5 μM for the isoquercitrin, quercetin and rutin, respectively. After consumption of quercitrin (quercetin 3-rhamnoside), they failed to detect any metabolites in the plasma. Gee et al., 2000 showed that isoquercitrin passes across the small intestinal epithelium more rapidly than free quercetin aglycone. These data established a ranking of flavonoid bioavailability as isoquercitrin>quercetin>rutin.

Naringinase is an enzyme preparation that can be produced from cultures of Penicillium aspergillus, Coniella diplodiella, Cochliobolus miyabeanus, Rhizoctonia solanti, Phomopsis citri, and Penicillium decumbens. Most commercial naringinase preparations were produced from Penicillium decumbens. Narikawa et al. (1998) concluded that Penicillium decumbens does degrade rutin, but their work was qualitative in nature, and they did not indicate what the results of that degradation were.

Naringinase is used to hydrolyze narigin, 7-(2-rhamnoside-beta-glucoside) of 4′,5,7-trihydroxyflavonone, to narigenin. It is used commercially to reduce the bitter taste in citrus fruit or juice. Naringinase was used by Uyeta et al. (1981) during an investigation of tea infusions. The effect of naringinase treatment on the mutagenic activity of tea infusions was similar to that of treatments with acid or hesperidinase. However, they neither characterized nor identified the hydrolyzed products. They did identify kaempferol, quercetin and myricetin as mutagenic principles in tea infusions treated with human fecal bacteria.

Although isoquercitrin would appear to be the most desirable quercetin derivative, there are currently no concentrated or pure forms of this compound available in the market place—other than very small amounts for use as analytical standards. There is no previously disclosed method for the processing of buckwheat foliar material for recovery of flavonoids and for the further biotransformation of such flavonoids to highly bioavailable, performance-enhanced, high-value products such as isoquercitrin and quercetin. Previously disclosed are only classical laboratory procedure for the extraction and purification of rutin.

Usually, the concentrations of naturally occurring isoquercitrin and quercetin found in biological systems are much lower than that of rutin. Isoquercitrin and quercetin extracted from biological systems demand much higher prices due to their rareness and bioavailability. There is not presently any commercially feasible technology for the biotransformation of rutin (regardless of the source) to highly bioavailable, performance enhanced and high value products such as isoquercitrin and quercetin.

### SUMMARY OF THE INVENTION

It is an object of this invention to provide an isoquercitrin-enriched composition derived from rutin, and to provide such a composition economically in commercial amounts sufficient to permit their use in functional foods, nutraceutical, natural health products, cosmetics and pharmaceutical applications.

It is a further object of this invention to provide a composition derived from rutin that is enriched in controlled proportions of isoquercitrin and quercetin, and to provide such a composition in commercial amounts sufficient to permit their use in functional foods, nutraceutical, natural health products, cosmetics and pharmaceutical applications.

It is a further object of this invention to provide a method whereby the yield of isoquercitrin can be maximized by inhibiting the conversion of isoquercitrin to quercetin. In the invention this is accomplished by the addition of an inhibitor of the β-D-glucosidase activity present in naringinase preparations.

It is a further object of this invention to provide a process for deriving rutin from buckwheat, and in particular to provide such a process deriving rutin from the buckwheat plant residue that remains in the field after buckwheat seed has been harvested, thereby converting a cheap waste product into a more valuable product.

In a first aspect the present invention provides a process for preparing a rutin enriched composition from biomass containing rutin, the process comprising performing a flavonoid extraction process on the biomass using an aqueous solution; filtering the solution to produce an extract solution; allowing the extract solution to stand such that a precipitate forms; collecting and drying the precipitate to form the rutin enriched composition.

Preferably the aqueous solution is maintained at a temperature above 30° C. during the extraction process. Preferably the aqueous solution is an aqueous alcohol solution, with an alcohol concentration of greater than 20% alcohol by volume, and for best results between 50% and 100% alcohol by volume. The extract solution is preferably concentrated to about one fifth to one tenth its original volume, and then chilled while standing to facilitate citation.

With the process of the present invention, a rutin enriched composition having 70% rutin content by weight can be prepared through relatively simple wet chemistry means and without the necessity of chromatographic means. Most economically the crop residue left after seeds have been harvested from a field of buckwheat is used to provide the rutin containing biomass. This residue has formerly had little if any value. Use of this crop residue is prefered over prior art use of buckwheat at the flowering stage since the seeds can be harvested, providing the primary return from a buckwheat crop. In the prior art the total return from a buckwheat crop is derived by purchasing it at the flowering, or other premature stage, as a feedstock for rutin production.

In a second aspect the invention provides a composition enriched in isoquercitrin prepared by a process comprising providing a solution having rutin suspended therein at conditions suitable for enzyme incubation; adding enzyme preparation comprising naringinase to the solution; maintaining the conditions of the solution suitable for enzyme incubation during an incubation period; terminating the incubation period by changing the conditions of the solution to conditions unsuitable for enzyme activity. These changes include lowering the pH and increasing the temperature of the solutione. Adjusting the duration of the incubation period controls the proportion of isoquercitrin in the composition.

In a third aspect the invention provides a composition enriched in isoquercitrin prepared by a process comprising providing a solution having rutin suspended therein at conditions suitable for enzyme incubation; adding an enzyme preparation comprising the enzymes naringinase or α-L-rhamnosidase to the solution; maintaining the conditions of the solution suitable for enzyme incubation during an incubation period; terminating the incubation period by changing the conditions of the solution to conditions unsuitable for enzyme incubation. For optimal yields, the temperature should be in the range of 50-55° C. and should not exceed 65° C. Adjusting the duration of the incubation period controls the proportion of isoquercitrin in the composition. The incubation period is optimally in the range of 1-48 hrs. Lowering pH and increasing the temperature of the solution terminates the incubation period by denaturing the enzyme preparation.

The proportion of isoquercitrin in the composition can be up to about 95%. The enzyme incubation with the enzyme preparation containing α-L-rhamnosidase and β-D-glucosidase also converts rutin to quercetin. The incubation period can be adjusted to provide a composition enriched with both isoquercitrin and quercetin in varying proportions.

Conveniently and economically the enzyme preparation can be naringinase, which is commercially available and economical. Naringinase is sold with a guaranteed content of the enzyme β-D-glucosidase for various commercial uses. Contrary to the prior art revealed by Narikawa et al (1998), it was found that naringinase from Penicillim decumbens was able to cleave sugar from the rutin.

Incubation of the enzyme α-L-rhamnosidase with rutin converts the rutin to isoquercitrin. Incubation of the enzyme β-D-glucosidase with isoquercitrin converts isoquercitrin to quercetin. Naringinase contains both the enzyme α-L-rhamnosidase and the enzyme β-D-glucosidase, and is commercially available in economic quantities.

An efficient, economic and commercially viable biotransformation can be accomplished without using purified or other expensive forms of α-L-rhamnosidase and β-D-glucosidase. Compositions with different ratios of rutin/isoquercitrin/quercetin can be tailor-made by manipulating biotransformation conditions. The process of the present invention produces a product of highly concentrated rutin, isoquercitrin quercetin, or mixtures thereof, which may then subsequently be purified using standard biochemical purification techniques.

In a fourth aspect of the invention, a β-D-glucosidase inhibitor is added to the rutin solution prior to the addition of the naringinase enzyme. In the preferred embodiment, the β-D-glucosidase inhibitor is D-Δ-gluconolactone. By inhibiting the β-D-glucosidase component of the naringinase preparation, isoquercitrin is not converted to quercetin; with the result that isquercitrin is obtained at high yield, and at purity greater than 80%.

The process of the present invention can be use to produce a product of highly concentrated rutin, isoquercitrin, quercetin or mixtures thereof from a variety of plant biomass sources, including, but not limited to members of the genus Fargopyrum, leaves of St. John's Wort ginkgo; biloba; alfalfa mulberry, algae; apple peel; pear peel; onion skin; asparagus tip; and rose pericarps.

The isoquercitrin-enriched product produce by the process of the present invention has bioactive properties including angiotensin-converting enzyme inhibitory, anti-inflammatory, antitumor, anti-viral, anti-oxidative, free radical scavenging, cancer preventative, cardioprotective, proteinase-inhibitory, protein kinase C inhibitory, tyrosine protein kinase inhibitory, topoisomerase II inhibitory and protein-cleaving enzyme inhibitory properties.

The bioactive properties of the isoquercitrin-enriched product produce by the process of the present invention will be useful as an additive in health foods, pharmaceuticals products, nutraceuticals and cosmetics. When added to products, the bioactive properties will be useful in the prevention and treatment of diseases and health problems, including, but not limited to cardiovascular disease, stroke, capillary fragility, arteriosclerosis, trauma, oxidative stress, hypertension, elevated cholesterol, elevated triglycerides, hyperglycemia, types II diabetes, obesity and related disorders, Alzheimer's disease, Parkinsonism, asthma and some cancers.

The present invention also offers processing and product flexibility enabling economical manufacture and satisfaction of market preferences.

These and other objects, features, and advantages of the invention become further apparent in the following detailed description of the invention that illustrates, by way of example, the principles of this invention.

### BRIEF DESCRIPTION OF THE DRAWINGS

While the invention is claimed in the concluding portions hereof, preferred embodiments are provided in the accompanying detailed description which may be best understood in conjunction with the accompanying diagrams where like parts in each of the several diagrams are labeled with like numbers, and where:

FIG. 1A illustrates the chemical structural formula for rutin;

FIG. 1B illustrates the chemical structural formula for isoquercitrin;

FIG. 1C illustrates the chemical structural formula for quercetin;

FIGS. 2A-2C show the results of HPLC analysis of: (2A) Methanol extract of buckwheat leaves (RT: 14.862-rutin, RT: 20.947=quercetin); (2B) Precipitate obtained from the aqueous alcohol extract of buckwheat leaves that had been concentrated to the aqueous phase and chilled (RT: 14.785=rutin). (2C) Conversion of rutin to isoquercitrin (RT: 15.181) and quercetin (RT: 20.372) after 24 hour incubation with naringinase. All samples were chromatographed on a C-18 Symmetry column eluted with a water:acetonitrile gradient containing 0.05% Trifluoroacetic acid. The column efluent was monitored at 280 nm and dissolved solids were quantified by ELSD.

FIGS. 3A-3C show the results of HPLC analysis of rutin samples: (3A) Commercial rutin sample. (Street Chemicals) RT: 14.875=rutin, RT: 15.442=isoquercitrin); (3B) Precipitate recovered after naringinase treatment of rutin (RT: 15.487=isoquercitin, RT: 20.843=quercetin); (3C) Purified isoquercitrin obtained by preparative HPLC (RT: 15.436=isoquercitrin). All samples were chromatographed on a C-18 Symmetry column eluted with a water acetonitrile gradient containing 0.05% Trifluoroacetic acid. The column effluent was monitored at 280 nm and dissolved solids were quantified by ELSD.

FIG. 4 is a summary flowchart of two methods of practicing the present invention. Using method A, rutin is recovered from plant biomass and then converted to a mixture of rutin, isoquercitrin and quercetin. Using method B, the addition of an inhibitor of β-D-glucosidase is added to prevent the conversion of isoquercitrin to quercetin. Using method B, the yield and purity of isoquercitrin by the method of the present invention is enhanced.

### DETAILED DESCRIPTION OF THE ILLUSTRATED EMBODIMENTS

The present invention provides a method for the production of high value bioavailable flavonoids from plant biomass. As described above, flavonoids have been shown to have a range of useful bioactive properties. One of the problems in the use of flavonoids in therapeutic applications is that they normally exist at low concentrations in nature. In order to use flavonoids as additives in pharmaceutical, nutraceutical or other health products, a method for purifying flavonoids is required.

In the present invention, the flavonoid rutin is recovered by standard biochemical methods. Rutin is then converted to isoquercitrin and quercetin through the action of the enzyme preparation naringinase. A further refinement of the present invention shows that the yield of the intermediate product isoquercitrin can be enhanced by selectively inhibiting the β-D-glucosidase activity present in the naringinase preparation, using the food additive d-Δ-gluconolactone.

The following examples and figures illustrate the operation of certain embodiments of the present invention so that it may be more readily understood.

With specific reference now to the figures in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. It is stressed that the particulars shown are by way of example and for the purposes of illustrative discussion,

Extraction of Rutin from Plant Biomass

Examples 1 and 2 below demonstrate that rutin in plant biomass can be recovered by a process involving extraction in aqueous solution, concentration and precipitation. It is contemplated that the step of concentrating the extract solution could be omitted, however that relatively simple and economical step increases the efficiency of the process.

As shown in Example 1, extraction in hot water as described recovered 36% of the available rutin from the leaves.

As shown in Example 2, exaction in an aqueous alcohol solution with 50% methanol by volume as described recovered 65% of the available rutin from the leaves.

As shown in Example 3, the rutin content of the rutin enriched composition can be increased to about 70% by simple wet chemistry means without using chromatography.

As shown in Example 4, the extraction efficiency of the process of the invention varies with the alcohol concentration, the temperature of the aqueous solution, the solid to solvent ratio, and the extraction time. For economic commercial processes, a suitable combination of these variables can be determined based on the economics of providing them.

It is anticipated that the extraction could be conducted in either a sequential batch or continuous feeding mode. The extraction recovery ratio of the process might also be improved by adding the extracted biomass to a fresh quantity of solvent and running second or additional extractions.

Prior art in this area was mostly focused on analytical methodology of flavonoids, concentration and quantity of flavonoids from biomass. Rutin enriched factions from precipitation have not earlier been described as a finished product. The value of a rutin-enriched composition has not earlier been recognized.

### EXAMPLE 1

### Aqueous Extraction, Concentration and Precipitation of Rutin from Buckwheat Leaf Material

Following harvest and drying, buckwheat leaves were prepared for extraction by grinding on a Wiley mill to pass a 2 mm screen. One kg of ground buckwheat leaves (rutin content is 3.74%, dry weight basis) were extracted in 10 L of water with continuous stirring at 90° C. for 1 hour. The resulting suspension was filtered, and the filtercake was washed 2 times with 300 ml of hot (95° C.) water. The wash filtrate was combined with the extract to give a combined extract volume of 8.6 L. The aqueous extraction procedure recovered 36% of the available rutin from the leaves. The extract was concentrated under reduced pressure to approximately ⅕ or 1/10 of the original volume. The concentrated extract was stored in the refrigerator (4° C.) overnight at which point the flavonoids precipitated out of solution. The precipitated material was collected following centrifugation at 7,000×g and filtration of the supernatant The pellet was subsequently freeze-dried. The rutin content of the precipitate was determined by dissolving an aliquot of the dried product in methanol and analyzing by RP-HPLC. From the HPLC results, we have concluded that 60% of the available rutin in the concentrated aqueous extract (reduced to ⅕ and 1/10 of original volume) can be recovered in the precipitate (pellet).

### EXAMPLE 2

### Aqueous Alcohol Extraction, Concentration and Precipitation of Rutin from Buckwheat Leaf Material

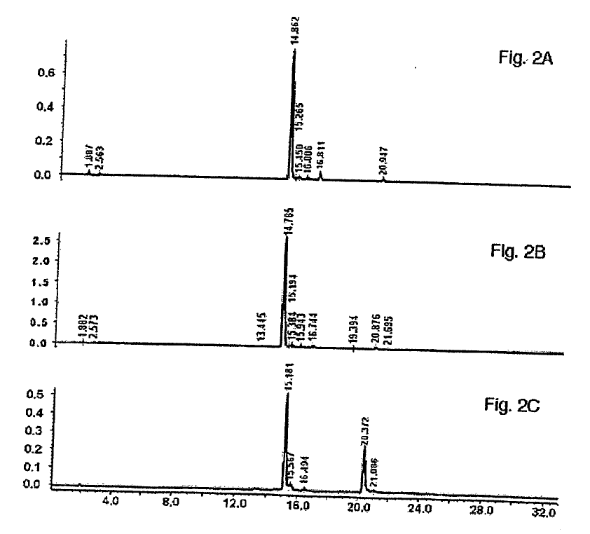

Following harvest and drying, buckwheat leaves were prepared for extraction by grinding on a Wiley mill to pass a 2 mm screen. One kg of ground buckwheat leaves (rutin content is 3.74%, dry weight basis) were extracted in 10 L of 50% (v/v) aqueous menthol with continuous stirring at 40° C. for 3 hours. The resulting suspension was filtered, and the filtercake was washed with warm (40° C.) 50% (v/v) aqueous methanol. The wash filtrate was combined with the extract The extraction procedure recovered 65% of the available rutin from the leaves. FIG. 2A illustrates the concentration of rutin in the methanol extraction. The extract was concentrated under reduced pressure to approximately ⅕ the original volume. The concentrated extract was stored in the refrigerator (4° C.) overnight at which point the flavonoids precipitated out of solution. The precipitated material was collected following centrifugation at 7,000×g and decantation of the supernatant. The precipitate was subsequently freeze-dried. The rutin content of the precipitate was determined by dissolving an aliquot of the dried product in methanol and analyzing by RP-HPLC. FIG. 2B illustrates the concentration of rutin in the precipitate. The flavonoid-enriched product was found to contain 64% rutin, and 6.88% protein. Rutin recovery of 93-100% was demonstrated in the precipitate (pellet) from the concentrated extract.

### EXAMPLE 3

### Purification of Rutin from the Intermediate Flavonoid Enriched Product Isolated from Buckwheat Leaves

The enriched rutin product from Example 2 was dissolved in warm methanol with vigorous stirring on a magnetic stirrer to facilitate complete solubilization of the rutin. Using vacuum filtration, any insoluble material was removed from the solution. The solution was evaporated to dryness at 40° C., under reduced pressure. The residue was then suspended in hot (90° C.) water with continuous stirring until most of the precipitate had dissolved. The suspension was allowed to precipitate in the refrigerator overnight. The precipitate was removed by vacuum filtration, and freeze-dried. The purified rutin precipitate was dissolved in methanol, filtered through a 0.45 um nylon syringe filter, and then analyzed by RP-HPLC to determine the purity of the product. Rutin content can be increased to about 70% or higher after repeat solubilization/crystallization without using chromatography.

### EXAMPLE 4

### Optimization of Rutin Extraction from Buckwheat Leaf Material

Buckwheat leaves obtained as noted in Example 2 were extracted with the following solvents in a Solid:Solvent ratio of 1:20 for 4 hours at 60° C.: Water, 30% (v/v) methanol/70% (v/v) water, 50% (v/v) methanol, 70% (v/v) methanol/ 30% (v/v) water, 85% (v/v) methanol/15% (v/v) water, and 100% methanol. The resulting extracts were then filtered and analyzed by RP-HPLC. The methanol content in the extraction solvent had a significant effect on the extraction efficiency of rutin from buckwheat leaves (Table 1.)

The optimal extraction conditions for the recovery of rutin from buckwheat leaves were determined from a series of optimization studies. The effects of varying the alcohol content of the extracting solvent, as well as the extraction temperature, extraction time and the solid to solvent ratio were significant. Tables 1-3 summarize some of these results

TABLE 1
Effect of the concentration of methanol in the extraction solvent on rutin
extraction efficiency using 1:20 solid:solvent ratio, and a 4 hour
extraction at 60° C.

| % methanol in solvent | Extraction Efficiency of Rutin |
|---|---|
| (%, v/v) | (%) |
| 0 | 1.0 |
| 30 | 29.2 |
| 50 | 86.5 |
| 70 | 94.1 |
| 85 | 83.7 |
| 100 | 85.7 |

[Extraction Efficiency of Rutin (%) = (total rutin in extract/total rutin in the starting material) × 100]

TABLE 2
Effect of extraction temperature on rutin extraction efficiency using 1:10
solid:solvent ratio, 70% (v/v) Methanol extraction solvent,
and a 4 hour extraction.

| Extraction Temperature | Extraction Efficiency of Rutin |
|---|---|
| (° C.) | (%) |
| 24 | 72.2 |
| 30 | 82.9 |
| 40 | 87.4 |
| 50 | 90.9 |
| 60 | 91.4 |

[Extraction Efficiency of Rutin (%) = (total rutin in extract/total rutin in the starting material) × 100]

TABLE 3
Effect of extraction time and solid:solvent ratio on rutin extraction
efficiency using 70% (v/v) Methanol extraction solvent at 50° C.

| | | Extraction Efficiency of |
|---|---|---|
| Solid:Solvent | Extraction Time | Rutin |
| Ratio | (hrs.) | (%) |
| 1:10 | 2 | 86.4 |
| 1:20 | 2 | 91.8 |
| 1:30 | 2 | 94.5 |
| 1:10 | 3 | 92.7 |
| 1:20 | 3 | 92.7 |
| 1:30 | 3 | 95.4 |
| 1:10 | 4 | 90.0 |
| 1:20 | 4 | 99.9 |
| 1:30 | 4 | 96.3 |

[Extraction Efficiency of Rutin (%) = (total rutin in extract/total rutin in the starting material) × 100]

Conversion of Rutin to Isoquercitrin and Quercetin

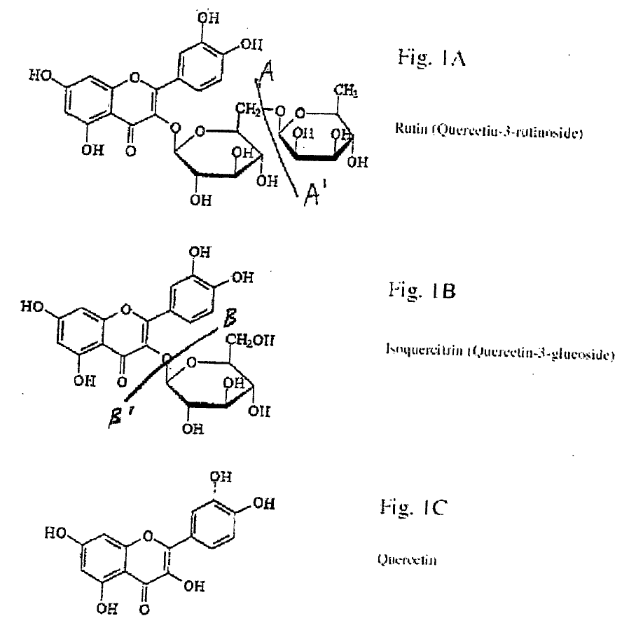

FIG. 1A illustrates the molecular make-up of rutin. Reaction of the enzyme α-L-rhamnosidase causes a biotransformation from rutin to the isoquercitrin of FIG. 1B by removing the first sugar on the bottom right hand side. To illustrate, the enzyme α-L-rhamnosidase essentially makes a conceptual incision along line A-A′ in FIG. 1A.

Reaction of the enzyme β-D-glucosidase causes a biotransformation from the isoquercitrin of FIG. 1B to the quercetin of FIG. 1C by removing the sugar on the bottom right hand side in FIG. 1B. To illustrate, the enzyme β-D-glucosidase essentially makes a conceptual incision along line B-B′ in FIG. 1B. As shown in Example 5, a composition enriched in isoquercitrin is prepared from the rutin-enriched composition of Example 2 above. The process for doing so comprises providing a solution having rutin suspended therein at conditions suitable for enzyme incubation. These conditions in Example 5 include the temperature of the solution to 80° C., and adjusting the pH to 4. An enzyme preparation, namely food-grade naringinase enzyme powder, comprising the enzymes α-L-rhamnosidase and β-D-glucosidase is added to the solution. The conditions of the solution are maintained at those conditions suitable for enzyme incubation during an incubation period with the temperature of the solution at a temperature of 50° C. and with continuous stirring.

Changing the conditions of the solution to conditions unsuitable for enzyme incubation terminates the incubation period. In Example 5 this changing included adjusting the pH to 2.5 and then heating to 80° C. for ten minutes with continuous stirring.

As seen in Table 4, adjusting the duration of the incubation period controls the proportion of isoquercitrin in the isoquercitrin-enriched composition. The proportion of isoquercitrin increases as the incubation period lengthens with weight ratios of rutin/isoquercitrin/quercetin of 1.71:1:0.06 after 8 hours; 0.33:1:0.07 after 16 hours; and trace: 1:0.46 after 24 hours.

Thus after 24 hours, substantially all the rutin has been converted to isoquercitrin and quercetin. After 24 hours however, the composition comprises only approximately twice as much isoquercitrin as quercetin. Prior to this, at 16 hours for instance, the composition comprises approximately fourteen times as much isoquercitrin as quercetin, and three times as much isoquercitrin as rutin.

As the incubation period increases further it can be seen that the isoquercitrin is further converted to quercetin, with the proportion of quercetin to isoquercitrin increasing until at 96 hours, the weight ratios of rutin/isoquercitrin/quercetin in the composition is trace: 1:3.38, and the composition comprises well over three times as much quercetin as isoquercitrin.

It can be readily seen that by adjusting the incubation period the proportions of rutin, isoquercitrin, and quercetin can be adjusted. The incubation times are measured in hours, such that considerable time latitude is available, allowing for conversion on a large scale in commercially significant quantities.

As shown in Example 6, after one day of enzymatic transformation, commercially sourced rutin (purity of 95% by weight) was converted to an isoquercitrin-enriched composition having weight ratios of rutin/isoquercitrin/quercetin of 0.1:1.0:0.2.

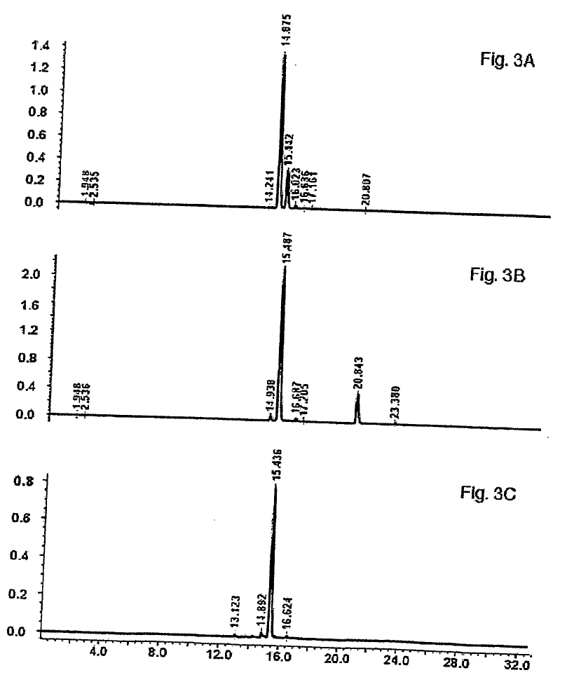

As shown in Example 7, commercial rutin was converted from the high rutin composition of FIG. 3A to the high isoquercitrin and quercetin composition of FIG. 3B.

As shown in Example 8, the high isoquercitrin and quercetin composition produced in Example 7 was further purified by Deltaprep C-18 chromatography, and high purity (95% +) isoquercitrin was obtained with a yield of 75% of the isoquercitrin in the starting material.

As shown in example 10, the β-glucosidase in the naringinase can be inhibited by the addition of D-Δ-gluconolactone, or other food facilitator, without affecting the activity of alpha-rhamnosidase. D-Δ-gluconolactone has been used for years as a food additive, for example as a coagulant in the production of tofu. In the present invention D-Δ-gluconolactone adds flexibility and further assurance that the process will produce high isoquercitrin yield. Selective inhibition of β-glucosidase, or selective separation of alpha-rhamnosidase from the naringinase for the production of isoquercitrin is within the scope of the claimed invention.

As shown in example 11, a medium-scale process is able to produce a highly enriched isoquercitrin product from buckwheat leaves. Thus, novel products can be produced from low value plant biomass.

Thus a commercially available enzyme mixture, naringinase is used to transform rutin into useful and highly valued flavonoids quercetin and isoquercitrin. The enzymatic transformation herein disclosed is efficient and less expensive than the prior art and does not utilize noxious and potentially harmful solvents. One of the biotransformed products generated from this study has rutin/isoquercitrin/quercetin weight ratios of trace: 22.8:7.3. Its composition is similar to the Ginkgo Biloba extract, which typically contains 24.5% flavone glycosides and 6.3% quercetin.

In conjunction with blending products processed under different conditions, products with different chemical profiles can be tailor-made. This technology offers some flexibility for making “designer-nutraceuticals.” Furthermore, the converted mixture could be fractionated and purified into high purity compounds using chromatography or other techniques.

Although chromatographic methods for the separation of flavonoids are described in the literature, they are principally designed for analytical purposes. The purification of enzyme-converted flavonoids (rutin, isoquercitrin and quercetin mixtures) using Stack Pack Columns, which would handle 5-50 liters of extract, is not previously known.

As shown in Example 9, the biotransformation technology disclosed by the invention also can convert rutin to isoquercitrin and quercetin St. John's Wort. Various other biomasses such as ginkgo biloba, alfalfa, mulberry leaves etc. as well as other rutin-enriched agricultural biomass such as rose hips, apple peels, pear peels, onion skins, and asparagus tips also contain rutin and could be used to produce the isoquercitrin-enriched composition.

Quercetin and isoquercitrin demand higher prices because of rareness and bioavailability/bioefficacy. The increased bioavailability of quercetin and isoquercitrin in cardiovascular disease and cancer prevention suggests a promising role of converted flavonoid product in the nutraceutical and pharmaceutical markets.

### EXAMPLE 5

### Conversion of Rutin to Isoquercitrin and Quercetin using Enzymatic Hydrolysis

By manipulating the biotransformation conditions, we were able to convert the flavonoid-enriched intermediates to products containing different profiles of rutin/isoquercitrin/quercetin.

The freeze-dried rutin product (approximately 60% Rutin) produced in Example 2 was used for the enzymatic conversion experiments. A quantity of 5 grams of dry rutin product was dispersed in 500 ml of water (solid:liquid ratio=1:100). The dispersion was heated to 80° C. and the pH adjusted to 4. The dispersion was then equilibrated at 50° C., followed by addition of food-grade naringinase enzyme powder (Amano Pharmaceutical Co., Ltd; Japan).

The naringinase preparation contains 150 units of beta-glucosidase or naringinase activity as described in the specifications from the supplier. A dosage of 66 mg of Amano naringinase was used per g of rutin in this trial. The enzymatic incubation was maintained at 50° C. with continuous stirring, for the appropriate length of time. Once the incubation time was complete, the enzyme was inactivated by adjusting the pH of the solution to 2.5 and then heating to 80° C. for 10 minutes with continuous stirring. After 10 minutes at 80° C., the solution was cooled to room temperature, and the pH adjusted to 7. The enzyme-converted product was then dried by spray drying, freeze drying or other appropriate means.

Table 4 summarizes the experimental conditions required to prepare products containing various rutin/isoquercitrin/quercetin profiles. The starting material described here was previously freeze-dried for convenience reason. The precipitate (pellet) recovered prior to the drying step in the Example 2 is also suitable as a starting material for the Example 5. The enzymatic conversion can be applied at different stages, i.e., prior to the extraction of flavonoids, after aqueous extraction, after pre-concentration, or after precipitation. Flavonoid profiles rutin/isoquercitrin/quercetin) remains unchanged in the control (without enzyme) following the identical procedures to the normal enzymatic treatment. This indicates at the transformation was caused by the action of naringinase.

TABLE 4
Enzymatic conversion of flavonoids with naringinase.

| | Enzyme Dosage | Incubation | Incubation | |
|---|---|---|---|---|
| | (mg enzyme/g Rutin | Time | Temperature | Weight Ratio of |
| Trial # | Ppt.) | (hrs.) | (° C.) | Rutin:Isoquercitrin:Quercetin |
| 1 | 66 | 8 | 50 | 1.71:1:0.06 |
| 2 | 66 | 16 | 50 | 0.33:1:0.07 |
| 3 | 66 | 24 | 50 | trace:1:0.46 |
| 4 | 66 | 48 | 50 | trace:1:0.72 |
| 5 | 66 | 72 | 50 | trace:1:1.65 |
| 6 | 66 | 96 | 50 | trace:1:3.38 |

### EXAMPLE 6

### Conversion of High Purity Commercial Rutin to Isoquercitrin

Using commercial rutin (95% Purity), purchased from Sigma Chemical Company, an enzyme incubation similar to that described in Example 5, was performed in order to convert the rutin to isoquercitrin. A quantity of 10.90 g of rutin was dispersed in 1000 water. The dispersion was heated to 80° C. and the pH adjusted to 4. The dispersion was then equilibrated at 55° C., followed by addition of 2.42 g of naringinase enzyme powder. The enzymatic incubation was maintained at 55° C. with continuous stirring, 24 hours. Once the incubation time was complete, the enzyme was inactivated by adjusting the pH of the solution to 2.5 and heating to 80° C. for 10 minutes with continuous stirring. After 10 minutes at 80° C., the solution was cooled to room temperature, and the pH adjusted to 7. A 1.0 ml aliquot of the extract was removed for RP-HPLC analysis of the composition of the final product. The remaining extract was freeze-dried. The HPLC results indicated that the pure rutin standard had been converted to a product containing a rutin/isoquercitrin/quercetin profile of 0.12:1:0.21 (weight ratio).

### EXAMPLE 7

### Scale-up Conversion

Commercial rutin purchased from Street Chemicals was used for the enzymatic conversion similar to that described in Example 6. Concentrations of rutin and isoquercitrin in the commercial rutin are shown in FIG. 3A. A quantity of 109 g of rutin was dispersed in 4000 ml water. The dispersion was heated to 80° C. and the pH adjusted to 4. The dispersion was then equilibrated at 55° C., followed by addition of 24.2 g of naringinase enzyme powder. The enzymatic incubation was maintained at 55° C. with continuous stirring, for 24 hours. Once the incubation time was complete, the enzyme was inactivated by adjusting the pH of the solution to 2.5 and heating to 80° C. for 10 minutes with continuous stirring. After 10 minutes at 80° C., the solution was cooled to room temperature, and the pH adjusted to 7. The solution was stored in the refrigerator (4° C.) overnight. The solids recovered from centrifugation were freeze-dried. A quantity of 61.8 g of dry matter was obtained. The chromatogram of this product is shown in the FIG. 3B.

### EXAMPLE 8

### Preparative Scale Isolation of Isoquercitrin and Quercetin

The solids (50 g) obtained from the method of example 7 were dissolved in 70% methanol and filtered. The resulting extract was subjected to preparative scale chromatography on a Waters reversed phase Bondapak C-18, 40×310 mm (15-20 125 Å) column eluted with a Methanol:1% acetic acid gradient at a flow rate of 50 ml/min using a Waters Delta-Prep 4000 system equipped with a 486 variable wavelength UV-Vis detector controlled by Millennium V 2.15 software. The compounds of interest were detected at 280 nm. The purity of the fractions collected were evaluated using the analytical HPLC procedure described in example A. The yield of isoquercitrin was 75% of the starting material with a 95% purity (FIG. 3C). A small quantity of pure quercetin was also recovered from one of the Preparative HPLC fractions. The purity of the preparative HPLC fractions could be further improved by re-crystallization from hot methanol.

### EXAMPLE 9

### Conversion of Rutin from St John's Wort Extract

The contents of several St. John's Wort capsules were combined, dispersed in water, and subjected to an enzyme incubation similar to that described in Example 5. St. John's Wort is known to contain rutin. The objective of this experiment was to convert the rutin present in the St. John's Wort extract to isoquercitrin. A quantity of 5.52 g of St. John's Wort extract was dispersed in 500 ml water. The dispersion was heated to 80° C. and the pH adjusted to 4. The dispersion was then equilibrated at 55° C., followed by addition of 0.60 g of naringinase enzyme powder. The enzymatic incubation was maintained at 55° C. with continuous stirring for 24 hours. Once the incubation time was complete, the enzyme was inactivated by adjusting the pH of the solution to 2.5 and heating to 80° C. for 10 minutes with continuous stirring. After 10 minutes at 80° C., the solution was cooled to room temperature, and the pH adjusted to 7. The extract was the freeze-dried. The dried product was dissolved in methanol, filtered and analyzed by RP-HPLC to determine the extent of the conversion of rutin to isoquercitrin. The HPLC results of the initial St. John's Wort extract indicated a rutin/isoquercitrin/quercetin profile of 0.47:1:0.21 (weight ratio). The enzyme converted product was found to contain a rutin/isoquercitrin/quercetin profile of trace: 1:0.18 (weight ratio) which indicated that all rutin present in the initial extract had been converted to isoquercitrin and quercetin.

### EXAMPLE 10

### Large-scale Conversion of Rutin to Isoquercitrin using Naringinase and D-delta-gluconolactone

Pharmaceutical grade rutin (38.15 g) from ICN was dispersed in 3.5 L of deionized water. Naringinase (8.47 g in 100 ml water) and D-Δ-gluconolactone (6.23 g in 100 ml water) solution were prepared. D-Δ-gluconolactone solution as added to the rutin:water mixture. The pH of the mixture was 4.0. The mixture was then heated to 80° C. and incubated 2 hr. The temperature was then reduced to 55° C. and the naringinase solution added. The mixture was incubated for 24 hr at 55° C. with stirring. To stop the reaction, the pH was decreased to 2.5 and the mixture heated to 80° C. for 10 min. The mixture was allowed to cool to room temperature and then the pH was adjusted to 7.0. The mixture was then refrigerated overnight to induce formation of a precipitate and the precipitate was allowed to settle. The precipitate was collected by centrigation and then freeze-dried (the PPT1 fraction). The supernatant fluid was concentrated and then re-centrifuged. The resultant pellet was also freeze-dried (the PPT2 fraction). Three batches were prepared in this manner. The rutin, isoquercitrin and quercetin in different fractions from each batch were analyzed by HPLC. The data are presented in Table 5.

The data in table 5 demonstrates that rutin and quercetin appear as minor components, whereas isoquercitrin is the principal product observed after enzymatic conversion. For example, a total of 77.72 g of isoquercitrin and 0.53 of quercetin were produced from the three batches that were processed by the method of example 10. The majority (61.8 g) of isoquercitrin appeared in the PPT 1 fraction. The conversion process was very efficient, as only 0.2009 g of rutin was left unconverted by the process. The data also shown that the inclusion of the inhibitor D-delta-gluconolactone to the reaction mixture selectively results in the production of primarily one type of flavonoid, namely isoquercitrin.

Isoquercitrin concentration in the PPT1 fraction varied from averaged 85.2% (the range was 81.1-88.8%). This example therefore, also shows that high purity (>80%) bioavailable flavinoids can be produced using simple biochemical purification techniques, and without the need for chromatographic methods. The supernatant (SUP) fractions were also valuable as they contained 14.42 g of isoquercitrin per 101.66 g of dry matter.

TABLE 5
Enzymatic conversion of flavinoids with naringinase and D-delta-
gluconolactone as a selective inhibitor.

| | Total Freeze- | Total | Total | Total |
|---|---|---|---|---|
| Batch # | dried weight (g) | Rutin (g) | Isoquercitrin (g) | Quercetin (g) |
| 1 | | | | |
| PPT1 | 23.23 | 0.0495 | 18.8495 | 0.5299 |
| PPT2 | 0.54 | 0.0054 | 0.2105 | — |
| SUP | 36.41 | 0.0844 | 6.9944 | — |
| 2 | | | | |
| PPT1 | 26.30 | 0.0604 | 23.3768 | — |
| PPT2 | 0.64 | — | 0.2828 | — |
| SUP | 30.64 | — | 2.3697 | — |
| 3 | | | | |
| PPT1 | 22.92 | — | 19.6154 | — |
| PPT2 | 1.34 | 0.0012 | 0.9727 | 0.0008 |
| SUP | 34.61 | — | 5.0559 | — |

### EXAMPLE 11

### Extraction, Conversion and Purification of Rutin from Buckwheat Leaf Materials

One kg of ManCan leaf material was extracted in 10 L of 70% methanol for 3 hr at 50° C. After 3 hr, the mixture was filtered and the plant material washed with approximately 4 L of hot 70% methanol. The filtrates were combined and the volume reduced using a rotary evaporator until the volume was ⅕ of the original volume. The concentrated extract was refrigerated and allowed to precipitate overnight. The mixture was then stirred, and then centrifuged to collect the rutin.

Based on previous analysis, rutin content was estimated to be 33.66 g from 1 kg of starting leaf material. The amounts of enzyme and inhibitor used were based on these estimates, and were similar to previous conversions (7.36 g naringinase; 6.23 g D-Δ-gluconolactone; 3.5 L water).

The rutin precipitate was added to 3.5 L of water, and D-Δ-gluconolactone solution added. The pH of the mixture was 4.0. The mixture was then heated to 80° C. and incubated for 2 hr. The mixture was then cooled to 55° C. and the naringinase solution added. The mixture was then incubated at 55° C. for 24 hrs. The reaction was stopped by reducing the pH to 2.5, and then incubating at 80° C. for 10 min. The mixture was cooled to room temperature and the pH adjusted to 7.0. The mixture was then placed at 4° C. overnight to allow a precipitate to form. The precipitate was collected by centrifugation as in Example 10.

The precipitate pellet was dissolved in methanol at 55° C. with stirring. The solution was filtered to remove insoluble material. The filtrate was then concentrated as much as possible without allowing the mixture to bubble in the concentration vessel. At this point, 1.5 L of hot water was added to the mixture, and the material re-precipitated by incubation at 4° C. for 2 days, and the precipitate collected by centrifugation. The re-precipitated material was then washed with hot water and precipitated for a third time. This final precipitate was freeze-dried to form a final product.

Note Respecting Methods and Examples

The buckwheat flavonoid content was determined by reverse phase high performance liquid chromatography (RP-HPLC) on a Waters Symmetry C-18 column (3.0×150 mm, 5 micrometer) eluted with a linear gradient of aqueous 0.05% v/v trifluoracetic acid (TFA): acetonitrile (T=0 min., % acetonitrile=10; T20 min, % acetonitrile=40; T-30 min., % acetonitrile=10) at a flow rate of 0.4 mL/min, with photodiode array (PDA) detector at 350 nm. Quantification of flavonoids was by external star curves using rutin, isoquercitrin and quercetin standards purchased commercially.

Rutin in the biomass was extracted out by solvent and determined by the HPLC method as described by Minami et al (1998). One gram of biomass (passed through a 100 mesh screen) was extracted with 40 ml of methanol at 70° C. for 60 min. in a Soxhelt extraction apparatus. The supernatant after centrifugation was used for the determination.

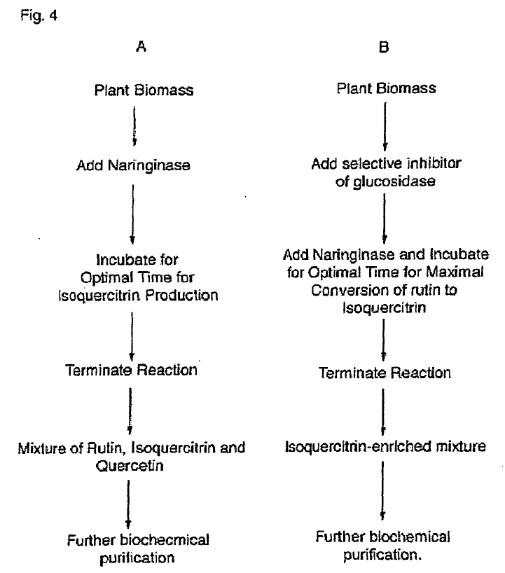

As summarized in FIG. 4, the present invention provides for the extraction, concentration, and precipitation of rutin enriched compositions from rutin containing plant biomass, and enzymatic conversion of rutin to isoquercitrin/quercetin enriched compositions using method A, or alternatively, the production of a isoquercitrin-enriched product. Both the products of method A or B of the invention are useful as additives for the health food, nutraceutical pharmaceutical, cosmetic and other markets.

The foregoing is considered as illustrative only of the principles of the invention. Further, since numerous changes and modifications will readily occur to those skilled in the art, it is not desired to limit the invention to the exact construction and operation shown and described, and accordingly, all such suitable changes or modifications in structure or operation which may be resorted to are intended to fall within the scope of the claimed invention.

We claim:

1. An isoquercitrin-enriched composition prepared by a process comprising:
providing a solution having rutin suspended therein at conditions suitable for enzyme incubation;
adding an enzyme preparation comprising naringinase to the solution;
maintaining the conditions of the solution suitable for enzyme incubation during an incubation period;
terminating the incubation period by changing the conditions of the solution to conditions unsuitable for said enzyme incubation;
wherein the proportion of isoquercitrin in the composition is controlled by adjusting the duration of the incubation period.

2. The isoquercitrin-enriched composition of claim 1 wherein the composition is further enriched with quercetin as a result of the enzyme incubation.

3. The isoquercitrin-enriched composition of claim 2 wherein the relative proportion of quercetin and isoquercitrin is controlled by adjusting the duration of the incubation period.

4. The isoquercitrin-enriched composition of claim 2 wherein the duration of the incubation period is dependent on the activity of the enzyme preparation.

5. The isoquercitrin-enriched composition of claim 2 wherein the duration of the incubation period is in the range of 1-48 hr.

6. The isoquercitrin-enriched composition of claim 1 wherein the conditions of the solution during enzyme incubation include temperature and pH level.

7. The isoquercitrin-enriched composition of claim 6 wherein the temperature is in the range of 50-55° C.

8. The isoquercitrin-enriched composition of claim 6 wherein the pH is in the range of 4-8.

9. The iwoquercitrin-enriched composition of claim 1 wherein the conditions of solution are an acidic pH and a temperature of substantially 80° C.

10. The isoquercitrin-enriched composition of claim 1 wherein the ratio of rutin to isoquercitrin is less than 20:1 by weight.

11. The isoquercitrin-enriched composition of claim 2 wherein the ratio of quercetin to isoquercitrin is greater than 0.003:1 by weight.

12. The isoquercitrin-enriched composition of claim 1 wherein said process further comprises purification of said solution following termination of said incubation period.

13. The isoquercitrin-enriched composition of claim 12 wherein the purification of said solution following termination of said incubation period is conducted using conventional biochemical purification.

14. A purified isoquercitrin composition comprising at least 90% isoquercitrin by weight prepared by subjecting the isoquercitrin-enriched composition of claim 1 to conventional biochemical purification methods.

15. The isoquercitrin-enriched composition of claim 1 wherein the rutin is obtained in enriched or purified form from a commercial source.

16. The isoquercitrin-enriched composition of claim 1 wherein the rutin is obtained by the method of claim 53.

17. An isoquercitrin-enriched composition containing isoquercitrin produced in accordance with the process of claim 1, said composition having bioactive properties comprising angiotensin-converting enzyme inhibitory, anti-inflammatory, anti-tumor, anti-viral, anti-oxidative, free radical scavenging, cancer preventative, cardioprotective, proteinase-inhibitory, protein kinase C inhibitory, tyrosine protein kinase inhibitory, topoisomerase II inhibitory and protein-cleaving enzyme inhibitory properties.

18. The isoquercitrin-enriched composition of claim 17, wherein the bioactive properties of said composition are used in the prevention and treatment of diseases and health problems, including, but not limited to cardiovascular disease, stroke, capillary fragility, arteriosclerosis, trauma, oxidative stress, hypertension, elevated cholesterol, elevated triglycerides, hyperglycemia, types II diabetes, obesity and related disorders, Alzheimer's disease, Parkinsonism, asthma and some cancers.

19. The isoquercitrin-enriched composition of claim 17, wherein said composition is used in functional foods.

20. The isoquercitrin-enriched composition of claim 17, wherein said composition is used in natural health products.

21. The isoquercitrin-enriched composition of claim 17, wherein said composition is used in nutraceutical products.

22. The isoquercitrin-enriched composition of claim 17, wherein said composition is used in pharmaceutical products.

23. The isoquercitrin-enriched composition of claim 17, wherein said composition is used in cosmetic products.

24. An isoquercitrin-enriched composition prepared by a process comprising:
providing a solution having rutin suspended therein at conditions suitable for enzyme incubation;
adding an enzyme preparation comprising naringinase to the solution;
maintaining the conditions of the solution suitable for enzyme incubation during an incubation period;
terminating the incubation period by changing the conditions of the solution to conditions unsuitable for enzyme incubation;
wherein the proportion of isoquercitrin in the composition is controlled by adjusting the duration of the incubation period.

24. The isoquercitrin-enriched composition of claim 24, wherein the yield of isoquercitrin is controlled by adjusting the duration of the incubation period.

25. The method of claim 24, wherein the duration of the incubation period is in the range of 1-48 hr.

26. The isoquercitrin-enriched composition of claim 24 wherein the relative proportion of rutin, quercetin and isoquercitrin is controlled by the addition of a β-D-glucosidase inhibitor to the solution.

27. The isoquercitrin-enriched composition of claim 26 wherein said β-D-glucosidase inhibitor is added to the solution before the addition of said enzyme preparation to the solution.

28. The isoquercitrin-enriched composition of claim 26 wherein the β-D-glucosidase inhibitor has the properties of D-Δ-gluconolactone.

29. The isoquercitrin-enriched composition of claim 28 wherein the β-D-glucosidase inhibitor is D-Δ-gluconolactone.

30. The isoquercitrin-enriched composition of claim 24 wherein the enzyme preparation comprises α-L-rhamnosidase.

31. The isoquercitrin-enriched composition of claim 24 wherein the conditions of the solution during enzyme incubation include temperature and pH level.

32. The isoquercitrin-enriched composition of claim 31 wherein the temperature is in the range of 50-55° C.

33. The isoquercitrin-enriched composition of claim 31 wherein the pH is in the range of 4-8.

34. The isoquercitrin-enriched composition of claim 24 wherein the conditions of the solution during enzyme incubation includes the addition of a β-D-glucosidase inhibitor.

35. The isoquercitrin-enriched composition of claim 34, wherein the β-D-glucosidase inhibitor has the properties of D-Δ-gluconolactone.

36. The isoquercitrin-enriched composition of claim 25 wherein the β-D-glucosidase inhibitor is D-Δ-gluconolactone.

37. The method of claim 35 wherein the concentration of D-Δ-gluconolactone is greater than 1 mM.

38. The isoquercitrin-enriched composition of claim 24 further comprising terminating the incubation period by denaturing the enzyme α-L-rhamnosidase.

39. The isoquercitrin-enriched composition of claim 24 wherein the rutin is obtained in enriched or purified form from a commercial source.

40. The isoquercitrin-enriched composition of claim 24 wherein the rutin is obtained by the method of claim 53.

41. The isoquercitrin-enriched composition of claim 24 wherein the ratio of rutin to isoquercitrin is less than 20:1 by weight.

42. The isoquercitrin-enriched composition of claim 24 wherein the ratio of quercetin to isoquercitrin is greater the 0.003:1 by weight.

43. The isoquercitrin-enriched composition of claim 24 wherein said process further comprises purification of said solution following termination of said incubation period.

44. The isoquercitrin-enriched composition of claim 43 wherein the purification of said solution following termination of said incubation period is conducted using conventional biochemical purification.

45. A purified isoquercitrin composition comprising at least 90% isoquercitrin by weight prepared by subjecting the isoquercitrin-enriched composition of claim 24 to conventional biochemical purification.

46. An isoquercitrin-enriched composition containing isoquercitrin produced in accordance with the process of claim 1, said composition having bioactive properties comprising angiotensin-converting enzyme inhibitory, anti-inflammatory, anti-tumor, anti-viral, anti-oxidative, free radical scavenging, cancer preventative, cardioprotective, proteinase-inhibitory, protein kinase C inhibitory, tyrosine protein kinase inhibitory, topoisomerase II inhibitory and protein-cleaving enzyme inhibitory properties.

47. The isoquercitrin-enriched composition of claim 46, wherein the bioactive properties of said composition are used in the prevention and treatment of diseases and health problems, including, but not limited to cardiovascular disease, stroke, capillary fragility, arteriosclerosis, trauma, oxidative stress, hypertension, elevated cholesterol, elevated triglycerides, hyperglycemia, types II diabetes, obesity and related disorders, Alzheimer's disease, Parkinsonism, asthma and some cancers.

48. The isoquercitrin-enriched composition of claim 46, wherein said composition is used in functional foods.

49. The isoquercitrin-enriched composition of claim 46, wherein said composition is used in natural health products.

50. The isoquercitrin-enriched composition of claim 46, wherein said composition is used in nutraceutical products.

51. The isoquercitrin-enriched composition of claim 46, wherein said composition is used in pharmaceutical products.

52. The isoquercitrin-enriched composition of claim 46, wherein said composition is used in cosmetic products.

53. A process for preparing a rutin enriched composition from biomass containing rutin, the process comprising:
performing a flavonoid extraction process on the biomass using an aqueous solution comprising water or alcohol;
filtering the solution to produce an extract solution;
allowing the exact solution to stand such that a precipitate forms;
collecting and drying the precipitate to form the rutin enriched composition.

54. The process of claim 53 wherein the flavonoid extraction process comprises fragmenting the biomass and agitating it in the aqueous solution.

55. The process of claim 53 further comprising concentrating the extract solution to form a concentrated act solution having less than one fifth of its original volume prior to allowing the extract solution to stand.

56. The process of claim 55 wherein the concentrated extract solution is allowed to stand at a temperature less than 10° C.

57. The process of claim 53 where the aqueous solution comprises water and is maintained at a temperature above 30° C.

58. The process of claim 53 who the aqueous solution comprises alcohol.

59. The process of claim 58 wherein the aqueous solution has a concentration of greater than 20% alcohol by volume, and the balance of the solution is water.

60. The process of claim 59 wherein the aqueous solution has a concentration of between 50% and 100% alcohol by volume, and the balance of the solution is water.

61. The process of claim 59 wherein the temperature of the aqueous solution is maintained at between 30° C. and 99° C. during the extraction process.

62. The process of claim 53 wherein the plant biomass comprises biomass from a member of the genus of Fargopyrum.

63. The process of claim 53 wherein the biomass comprises at least one of leaves of St. John's Wort; ginkgo; biloba; alfalfa; mulberry; algae; apple peels; pear peels; onion skins; asparagus tips; and rose hip pericarps.

64. A flavonoid-enriched composition containing rutin produced in accordance with the process of claim 53, said composition having bioactive properties comprising angiotensin-converting enzyme inhibitory, anti-inflammatory, anti-tumor, anti-viral, anti-oxidative, free radical scavenging, cancer preventative, cardioprotective, proteinase-inhibitory, protein kinase C inhibitory, tyrosine protein kinase inhibitory, topoisomerase II inhibitory and protein-cleaving enzyme inhibitory properties.

65. The flavonoid-enriched composition of claim 64, wherein the bioactive properties of said composition are used in the prevention and treatment of diseases and health problems, including, but not limited to cardiovascular disease, stroke, capillary fragility, arteriosclerosis, trauma, oxidative stress, hypertension, elevated cholesterol elevated triglycerides, hyperglycemia, types II diabetes, obesity and related disorders, Alzheimer's disease, Parkinsonism, asthma and some cancers.

66. The flavonoid-enriched composition of claim 64, wherein said composition is used in functional foods.

67. The flavonoid-enriched composition of claim 64, wherein said composition is used in natal health products.

68. The flavonoid-enriched composition of claim 64, wherein said composition is used in nutraceutical products.

69. The flavonoid-enriched composition of claim 64, wherein said composition is used in pharmaceutical products.

70. The flavonoid-enriched composition of claim 64, wherein said composition is used in cosmetic products.

71. A method of producing an isoquercitrin-enriched composition, said method comprising:
providing a solution having rutin suspended therein at conditions suitable for enzyme incubation;
adding an enzyme preparation comprising naringinase to the solution;
maintaining the conditions of the solution suitable for enzyme incubation during an incubation period;
terminating the incubation period by changing the conditions of the solution to conditions unsuitable for said enzyme incubation, said solution at this point being an isoquercitrin-enriched composition;
wherein the proportion of isoquercitrin in the composition is controlled by adjusting the duration of the incubation period.

72. The method of claim 71 wherein said composition also contains quercetin as a result of said enzyme incubation.

73. The method of claim 72 wherein the relative proportion of quercetin and isoquercitrin is controlled by adjusting the duration of the incubation period.

74. The method of claim 71 wherein the duration of the incubation period is dependent on the activity of the enzyme preparation.

75. The method of claim 71 wherein the duration of the incubation period is in the range of 1-48 hr.

76. The method of claim 71 wherein the conditions of the solution during enzyme incubation include temperature and pH level.

77. The method of claim 76 wherein the temperature is in the range of 50-55° C.

78. The method of claim 76 wherein the pH is in the range of 4-8.

79. The method of claim 71 wherein the conditions of solution are an acidic pH and a temperature of substantially 80° C.

80. The method of claim 71 wherein the ratio of rutin to isoquercitrin is less than 20:1 by weight.

81. The method of claim 80 wherein the ratio of quercetin to isoquercitrin is greater than 0.003:1 by weight.

82. The method of claim 71 further comprising purification of said solution following termination of said incubation period.

83. The method of claim 82 wherein the purification of said solution following termination of said incubation period is conducted using conventional biochemical purification.

84. The product, purified isoquercitrin, manufactured by the method of claim 83.

85. An isoquercitrin-enriched composition containing isoquercitrin produced in accordance with the method of claim 71, said composition having bioactive properties comprising angiotensin-converting enzyme inhibitory, anti-inflammatory, anti-tumor, anti-viral, anti-oxidative, free radical scavenging, cancer preventative, cardioprotective, proteinase-inhibitory, protein kinase C inhibitory, tyrosine protein kinase inhibitory, topoisomerase II inhibitory and protein-cleaving enzyme inhibitory properties.

86. The isoquercitrin-enriched composition of claim 85, wherein the bioactive properties of said composition are used in the prevention and treatment of diseases and health problems, including, but not limited to cardiovascular disease, stroke, capillary fragility, arteriosclerosis, trauma, oxidative stress, hypertension, elevated cholesterol, elevated triglycerides, hyperglycemia, types II diabetes, obesity and related disorders, Alzheimer's disease, Parkinsonism, asthma and some cancers.

87. The isoquercitrin-enriched composition of claim 85, wherein said composition is used in functional foods.

88. The isoquercitrin-enriched composition of claim 85, wherein said composition is used in natural health products.

89. The isoquercitrin-enriched composition of claim 85, wherein said composition is used in nutraceutical products.

90. The isoquercitrin-enriched composition of claim 85, wherein said composition is used in pharmaceutical products.

91. The isoquercitrin-enriched composition of claim 85, wherein said composition is used in cosmetic products.

92. A method of producing an isoquercitrin-enriched composition, said method comprising:
providing a solution having rutin suspended therein at conditions suitable for enzyme incubation;
adding an enzyme preparation comprising naringinase to the solution;
maintaining the conditions of the solution suitable for enzyme incubation during an incubation period;
terminating the incubation period by changing the conditions of the solution to conditions unsuitable for enzyme incubation;
wherein the proportion of isoquercitrin in the composition is controlled by adjusting the duration of the incubation period.

93. The method of claim 92, wherein the yield of isoquercitrin is controlled by adjusting the duration of the incubation period.

94. The method of claim 92, wherein the duration of the incubation period is in the range of 1-48 hr.

95. The method of claim 92 further comprising the addition of a β-D-glucosidase inhibitor to the solution to control the relative proportion of rutin, quercetin and isoquercitrin in the solution.

96. The method of claim 95 wherein said β-D-glucosidase inhibitor is added to the solution before the addition of said enzyme preparation to the solution.

97. The method of claim 95 wherein the β-D-glucosidase inhibitor has the properties of β-D-gluconolactone.

98. The method of claim 97 wherein the β-D-glucosidase inhibitor is D-Δ-gluconolactone.

99. The method of claim 92 wherein the enzyme preparation on comprises α-L-rhamnosidase.

100. The method of claim 92 wherein the conditions of the solution during enzyme incubation include temperature and pH level.

101. The method of claim 100 wherein the temperature is in the range of 50-55° C.

102. The method of claim 100 wherein the pH is in the range of 4-8.

103. The method of claim 92 wherein the conditions of the solution during enzyme incubation includes the addition of a β-D-glucosidase inhibitor.

104. The method of claim 103, wherein the β-D-glucosidase inhibitor has the properties of D-Δ-gluconolactone.

105. The method of claim 104 wherein the β-D-glucosidase inhibitor is D-Δ-gluconolactone.

106. The method of claim 105 wherein the concentration of D-Δ-gluconolactone is greater than 1 mM.

107. The method of claim 92 further comprising terminating the incubation period by denaturing the enzyme β-L-rhamnosidase.

108. The method of claim 92 wherein the ratio of rutin to isoquercitrin is less than 20:1 by weight.

109. The method of claim 92 wherein the ratio of quercetin to isoquercitrin is greater than 0.003:1 by weight.

110. The method of claim 92 further comprising purification of said solution following termination of said incubation period.

111. The method of claim 10 wherein the purification of said solution following termination of said incubation period is conducted using conventional biochemical purification.

112. The product, purified isoquercitrin, produced in accordance with the method of claim 111.

113. An isoquercitrin-enriched composition containing isoquercitrin produced in accordance with the process of claim 92, said composition having bioactive properties comprising angiotensin-converting enzyme inhibitory, anti-inflammatory, anti-tumor, anti-viral, anti-oxidative, free radical scavenging, cancer preventative, cardioprotective, proteinase-inhibitory, protein kinase C inhibitory, tyrosine protein kinase inhibitory, topoisomerase II inhibitory and protein-cleaving enzyme inhibitory properties.

114. The isoquercitrin-enriched composition of claim 113, wherein the bioactive properties of said composition are used in the prevention and treatment of diseases and health problems, including, but not limited to cardiovascular disease, stroke, capillary fragility, arteriosclerosis trauma, oxidative stress, hypertension, elevated cholesterol elevated triglycerides, hyperglycemia, types II diabetes, obesity and related disorders, Alzheimer's disease, Parkinsonism, asthma and some cancers.

115. The isoquercitrin-enriched composition of claim 113, wherein said composition is used in functional foods.

116. The isoquercitrin-enriched composition of claim 113, wherein said composition is used in natural health products.

117. The isoquercitrin-enriched composition of claim 113, wherein said composition is used in nutraceutical products.

118. The isoquercitrin-enriched composition of claim 113, wherein said composition is used in pharmaceutical products.

119. The isoquercitrin-enriched composition of claim 113, wherein said composition is used in cosmetic products.
